(19) United States
(12) Patent Application Publication
(10) Pub. No.: US 2008/0262378 A1
(43) Pub. Date: Oct. 23, 2008

(54) IMPLANTABLE THERAPY DELIVERY SYSTEM HAVING MULTIPLE TEMPERATURE SENSORS

(75) Inventors: Martin T. Gerber, Maple Grove, MN (US); John C. Rondoni, Plymouth, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 11/737,171

(22) Filed: Apr. 19, 2007

(51) Int. Cl. A61B 5/01

(52) U.S. Cl. 600/549

(57) ABSTRACT

A system includes an implantable medical device and an implantable therapy delivery element. The device includes a housing, electronics disposed in the housing, and a first temperature sensor operably coupled to the electronics and located in proximity to the housing. The element has a proximal end portion configured to be operably couplable to the device and a distal end portion configured to be placed in a patient at a location removed from the device. A second temperature sensor located on the element between the proximal end and the distal end. The second temperature sensor is operably couplable to the electronics. The electronics are configured to compare a value associated with the first temperature sensed by the first temperature sensor to a value associated with the second temperature sensed by the second temperature sensor. The system may be used for monitoring an infection in proximity to the device.

### FIELD

This disclosure relates, inter alia, to implantable medical devices. More particularly, it relates to systems, devices and methods that employ more than one temperature sensor to monitor infection or other potentially adverse conditions in proximity to an implantable medical device.

### BACKGROUND

Infection associated with implantation of medical devices is a serious health and economic concern. Today, infections associated with implanted medical devices are not very common due to care and precautions taken during surgical implantation of the devices. However, when infection associated with an implanted medical device (IMD) does occur, explanting the device is often the only appropriate course of action.

For IMDs having a battery powered component, such as implantable cardiac pacemakers, cardioverter/defibrillators having pacing capabilities, other electrical stimulators including spinal cord, deep brain, nerve, and muscle stimulators, infusion devices, cardiac and other physiologic monitors, cochlear implants, etc., the battery powered component is typically enclosed in a housing that is implanted subcutaneously at a surgically prepared site, referred to as a “pocket”. Associated devices, such as elongated medical electrical leads or drug delivery catheters, extend from the pocket to other subcutaneous sites or deeper into the body to organs or other implantation sites.

Surgical preparation and implantation are conducted in a sterile field, and the IMD components are packaged in sterile containers or sterilized prior to introduction into the sterile field. However, despite these precautions, there always is a risk of introduction of microbes into the pocket. Surgeons therefore typically apply disinfectant or antiseptic agents to the skin at the surgical site prior to surgery, directly to the site before the incision is closed, and prescribe oral antibiotics for the patient to ingest during recovery.

Despite these precautions, infections do occur. In addition, once the pocket becomes infected, the infection can migrate along the lead or catheter to the heart, brain, spinal canal or other location in which the lead or catheter is implanted. Such a migrating infection can become intractable and life-threatening, requiring removal of the IMD in the pocket and associated devices, such as leads and catheters. Removal of a chronically implanted lead or catheter can be difficult and dangerous. Accordingly, aggressive systemic drug treatment is prescribed to treat such infections. However, early detection of infection associated with implanted medical devices may allow for earlier intervention, resulting in fewer device explants.

Monitoring of infection through the use of temperature sensors has been proposed, but not in proximity to implantable medical devices that deliver therapy. Further, no adequate solution for reliably monitoring infection in proximity to such devices has been proposed.

### SUMMARY

The present disclosure describes, inter alia, systems, devices and methods that employ more than one temperature sensor to monitor infection or other potentially adverse conditions in proximity to an implantable medical device. At least one temperature sensor is located in proximity to an implanted therapy delivery device and at least one temperature sensor is located at a position removed from the therapy delivery device. The temperatures at the two locations may be compared to determine more accurately whether the temperature monitored in proximity to the therapy delivery device is indicative of infection.

In various embodiments, a system is described. The system includes an implantable medical device and an implantable therapy delivery element. The implantable medical device includes a housing, electronics disposed in the housing, and a first temperature sensor operably coupled to the electronics and located in proximity to the housing. The implantable therapy delivery element has a proximal end portion configured to be operably couplable to the implantable medical device and a distal end portion configured to be placed in a patient at a location removed from the implantable medical device. A second temperature sensor is located on the therapy delivery element between the proximal end and the distal end. The second temperature sensor is operably couplable to the electronics. The electronics are configured to compare a value associated with a first temperature sensed by the first temperature sensor to a value associated with a second temperature sensed by the second temperature sensor.

In various embodiments, a method for monitoring an infection in proximity to an implanted therapy delivering medical device is described. The method includes monitoring a first patient tissue temperature via a first temperature sensor located on a therapy delivering implanted medical device and monitoring a second patient tissue temperature via a second temperature sensor located on an associated therapy element at a location removed from the therapy delivering implanted medical device. The method further includes determining whether an infection is present in proximity to the therapy delivering medical device by comparing information relating to the first monitored temperature to information relating to the second monitored temperature. The method further includes issuing an alert if a determination is made that an infection is present in proximity to the therapy delivering medical device.

The present disclosure provides devices, systems and methods that allow for reliable monitoring of infection in proximity to implanted therapy delivery medical devices through the use of temperature sensing. By employing at least two temperature sensors, one in proximity to the implanted therapy delivery medical device and one at a location removed from the implanted therapy delivery medical device, information relating to temperature obtained from the two sensors can be compared to more accurately determine whether temperature in proximity to the device is indicative of a localized condition, such as a localized infection, or a more general condition of the patient. The systems described herein may be readily employed to carry out such comparisons. These and other advantages will be readily understood from the following detailed descriptions when read in conjunction with the accompanying drawings.

### BRIEF DESCRIPTION OF THE DRAWINGS

FIG. 1 is a diagrammatic representation of a side view of an implantable therapy delivery system.

FIG. 2 is a block view of an implantable therapy delivery system.

FIGS. 3A-B are diagrammatic representations of side views of implantable therapy delivery systems.

FIG. 4 is a diagrammatic representation of an exploded view of a representative therapy delivery system.

FIG. 5 is a diagrammatic representation of an environment of a representative spinal cord stimulation (SCS) system implanted in a patient.

FIG. 6 is a schematic block diagram of representative components of a representative implantable medical device.

FIG. 7 is block diagram of portions of a representative memory.

FIGS. 8-10 are flow diagrams of representative methods.

FIGS. 11A-D are schematic block diagrams of a representative implantable medical devices or systems.

The drawings are not necessarily to scale. Like numbers used in the figures refer to like components, steps and the like. However, it will be understood that the use of a number to refer to a component in a given figure is not intended to limit the component in another figure labeled with the same number.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “therapy delivery medical device”, or the like, means a medical device that provides a therapy, such as a therapeutic agent or an electrical signal, to a patient. Non-limiting examples of implantable therapy delivering devices include implantable infusion devices and implantable electrical signal generators, such as cardiac defibrillators, pacemakers, neurostimulators, gastric stimulators, and cochlear implants.

As used herein, “couplable” means capable of being coupled.

As used herein, “crosses a threshold”, or the like, means meets or exceeds a threshold. It will be understood that a decrease in a value may “exceed” a threshold.

As used herein, “comparable value” or the like, in the context comparing a first temperature to a second temperature, means a value of a type of data the same as the type of data to which it is being compared. For example, a value comparable to a temperature at a point in time is another temperature at a point in time. By way of further example, a value comparable to a percent deviation from a mean temperature is another percent deviation from a mean temperature.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

The recitation of numerical ranges by endpoints includes all numbers subsumed within that range (e.g. 1 to 5 includes 1, 1.5, 2, 2.75, 3, 3.80, 4, and 5) and any range within that range.

The present disclosure describes, inter alia, systems, devices and methods that employ more than one temperature sensor to monitor infection or other potentially adverse conditions in proximity to an implantable medical device. At least one temperature sensor is located in proximity to an implanted therapy delivery device and at least one temperature sensor is located at a position removed from the therapy delivery device. The temperatures at the two locations may be compared to determine whether the temperature monitored in proximity to the therapy delivering device is indicative of a potentially adverse condition, such as a localized infection, in proximity to the implanted therapy delivery device.

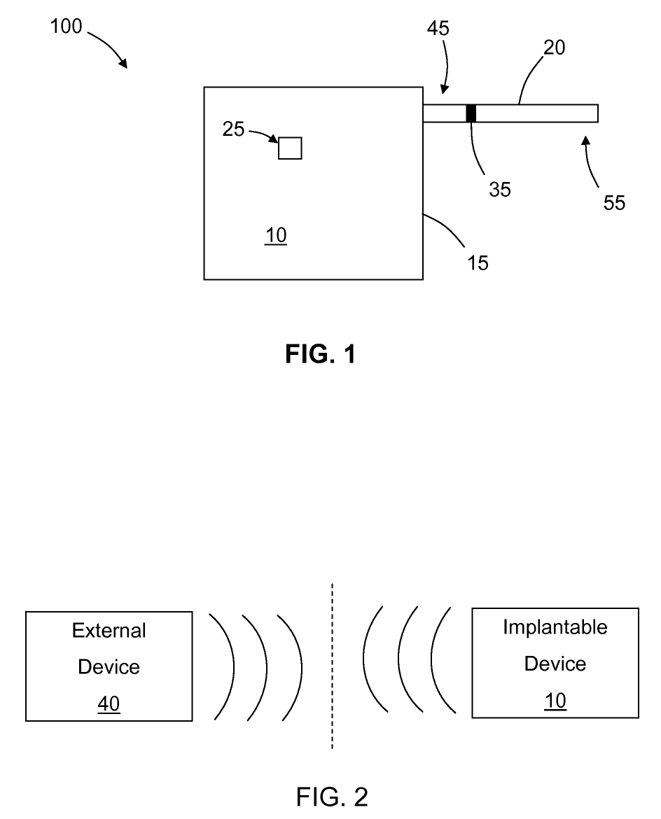

Referring to FIG. 1, a diagrammatic representation of an implantable therapy delivery system 100 is shown. The system 100 includes an implantable therapy delivery device 10 and an associated therapy element 20 operably coupled to therapy delivery device 10. Therapy delivery device 10 may be any device capable of delivering therapy to a patient. For example, therapy delivery device may be an infusion device or an electrical signal generator. Associated therapy delivery element 20 may be any element capable of delivering therapy from therapy device 10 to the patient, such as a catheter or a lead. Therapy delivery device 10 includes a housing 15 and a temperature sensor 25 located in proximity to housing 15. For example, temperature sensor 25 may be integrated with housing 15. If housing 15 is hermetically sealed, feedthroughs (not shown) may be used to provide electrical connectivity through housing 15 while maintaining the hermetic seal. Electronics (not shown in FIG. 1) are disposed in housing and temperature sensor 25 is operably coupled to the electronics. Electronics may also be configured to control delivery of therapy to a patient into which system 100 is implanted.

As further shown in FIG. 1, therapy delivery element comprises a proximal end portion 45 and a distal end portion 55. Proximal end portion 55 is configured to be operably coupable to therapy delivery device 10. Distal end portion 55 is typically configured to deliver therapy deliverable from device 10 to patient. A second temperature sensor 35 is located on therapy element 20 between the proximal end and the distal end of element 20. If second temperature sensor 35 is located at, e.g., the distal end, it will be considered as being located between the proximal and distal ends of element 20. When therapy element 20 is coupled to device 10, second temperature sensor 35 is operably couplable to electronics of device 10.

Temperature sensor 25, 35 may be any sensor capable of sensing temperature. For example, temperature sensor 25, 35 may include a thermocouple, a thermistor, a junction-based thermal sensor, a thermopile, a fiber optic detector, an acoustic temperature sensor, a quartz or other resonant temperature sensor, a thermo-mechanical temperature sensor, a thin film resistive element, or the like.

Referring to FIG. 2, an external device 40 in wireless communication with implantable therapy delivery device 10 is shown. According to various embodiments, external device 40 may communicate with implantable therapy delivery device 10 through patient's skin, which is represented by the dashed line in FIG. 2. In various embodiments, implantable device 10 carries out the various temperature comparisons and condition monitoring determinations, or portions thereof, described herein. In some embodiments, the combination of implantable device 10 and external device 40 carry out the various condition monitoring methods, or portions thereof, described herein. In various embodiments, where implantable therapy delivery device 10 is a programmable device, external device 40 may be a programmer device, such as Medtronic Inc.'s N'Vision™ clinician programmer. Of course external device may be any device capable of wirelessly communicating with implantable therapy delivery device 10, such as a patient programmer, a computer, a personal data assistant, or the like. External device 40 and implantable device 1 may be capable of one-way (external device 40 to implantable device 1 or implantable device 1 to external device 40) or two-way communication.

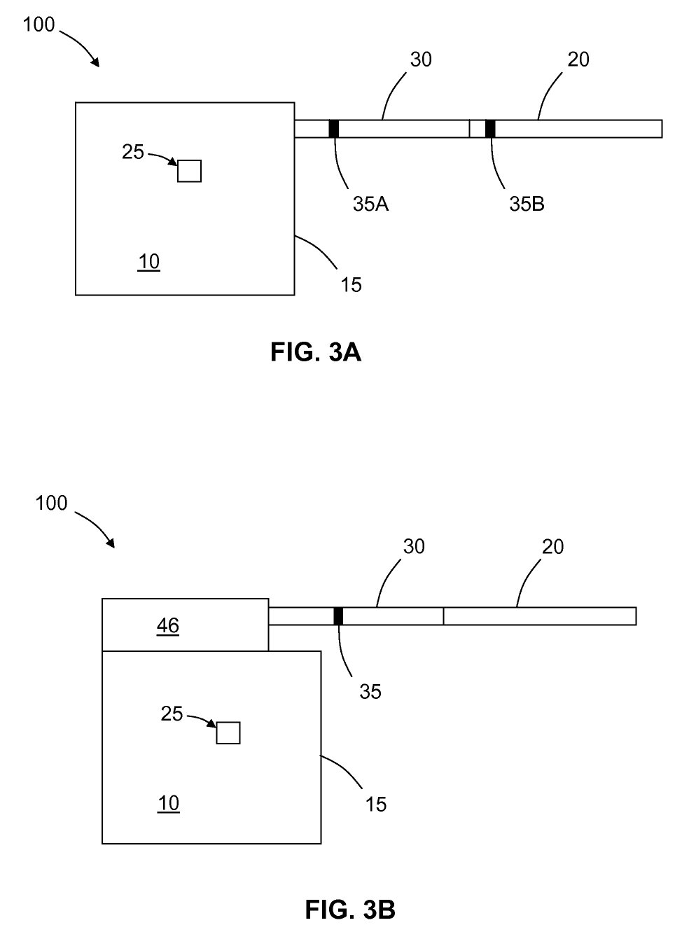

In the embodiments shown in FIG. 3, implantable therapy delivery device 10 is an electrical signal generator, such as Medtronic Inc.'s Restore™ Advanced implantable neurostimulator, and associated therapy delivery element 20 is a lead. As further shown in FIG. 3, such a system 100 may include a lead extension 30 or other adaptor to couple lead 20 to electrical signal generator 10. While not shown, it will be understood that more than one lead 20 may be operably coupled to one electrical signal generator device 10 or one extension 30 or that more than one extension 30 may be operably coupled to one electrical signal generator device 10. As shown in FIGS. 3A and B, one or more second temperature sensor 35, 35A, 35B may be located extension 30 or lead 20.

Referring to FIG. 3B, electrical signal generator device 10 may include a connector portion 46 for connecting to lead 20 or extension 30 or other adaptor to couple lead 20 to device 10. While not shown, it will be understood that lead 20 may be coupled to electrical signal generator 10 without extension 30 or adaptor.

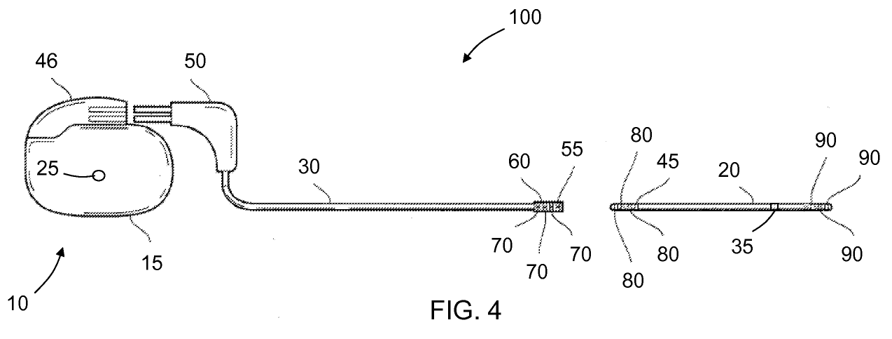

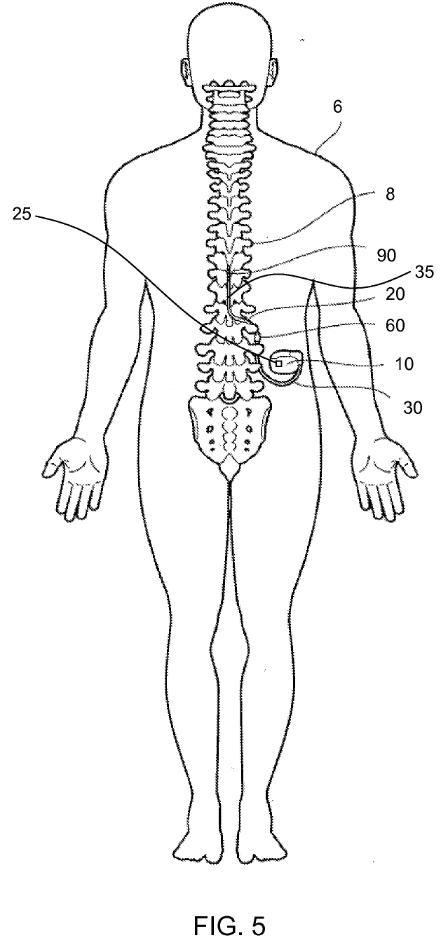

Referring to FIG. 4, an exploded view of a representative implantable electrical signal generator system 100 is shown. In the system shown in FIG. 5, electrical signal generator 10 comprises a connector block 46 configured to receive connector 50 at proximal end of extension 30. The distal end portion of extension 30 includes a connector 60 configured to receive proximal end of lead 20. Connector 60 comprises internal electrical contacts 70 configured to electrically couple extension 30 to lead 20 via electrical contacts 80 disposed on the proximal end portion of lead 20. Electrodes 90 are disposed on distal end portion of lead 20 and are electrically coupled to electrical contacts 80, typically through conductors (not shown). Lead 20 may include any number of electrodes 90, e.g. one, two, three, four, five, six, seven, eight, sixteen, thirty-two, or sixty-four. Typically, each electrode 90 is electrically coupled to a discrete electrical contact 80.

As shown in FIG. 4, connector 60 may also include an internal electrical contact 55 configured to contact electrical contact 45 of lead 20. Contact 45 is electrically coupled to second temperature sensor via a conductor (not shown) running through lead body. Connector 50 of extension 30 contains a contact (not shown) electrically coupled to contact 45 of connector 60 at distal end portion of extension 30. Connector block 46 may contain a contact (not shown) configured to electrically couple contact 45 to electronics of device 10. Of course, second temperature sensor 35 may be located on extension 30 and may similarly be electrically couplable to electronics of device 10, or lead 20 may be directly coupled to device 10 and contact 45 of lead 20 may be electrically couplable with contact. Any known of future developed suitable means for electrically coupling second temperature sensor 35 to electronics of device 10 may be used. While not shown, it will be understood that a catheter may be modified to include a contact and temperature sensor as described above and that an infusion pump may be modified to electrically couple the catheter temperature sensor to electronics of the infusion pump.

By way of example and referring to FIG. 5, a spinal cord stimulation (SCS) system, is shown implanted in a patient 6. For SCS, an implantable electrical signal generator 10 is typically placed in the abdominal region of patient 6 and lead 20 is placed at a desired location along spinal cord 8. Implantable electrical signal generator 10 is capable of generating electrical signals that may be applied to tissue of patient 6 via electrodes 90 for therapeutic or diagnostic purposes. IPG 10 contains a power source and electronics for sending electrical signals to the spinal cord 8 via electrodes 90 to provide a desired therapeutic effect. As shown in FIG. 5, a first temperature sensor 25 is located in proximity to device 10 and second temperature sensor 35 is located on lead 20, at a location removed from device 10. It will be appreciated that other systems employing other therapy delivery devices and associated devices are contemplated.

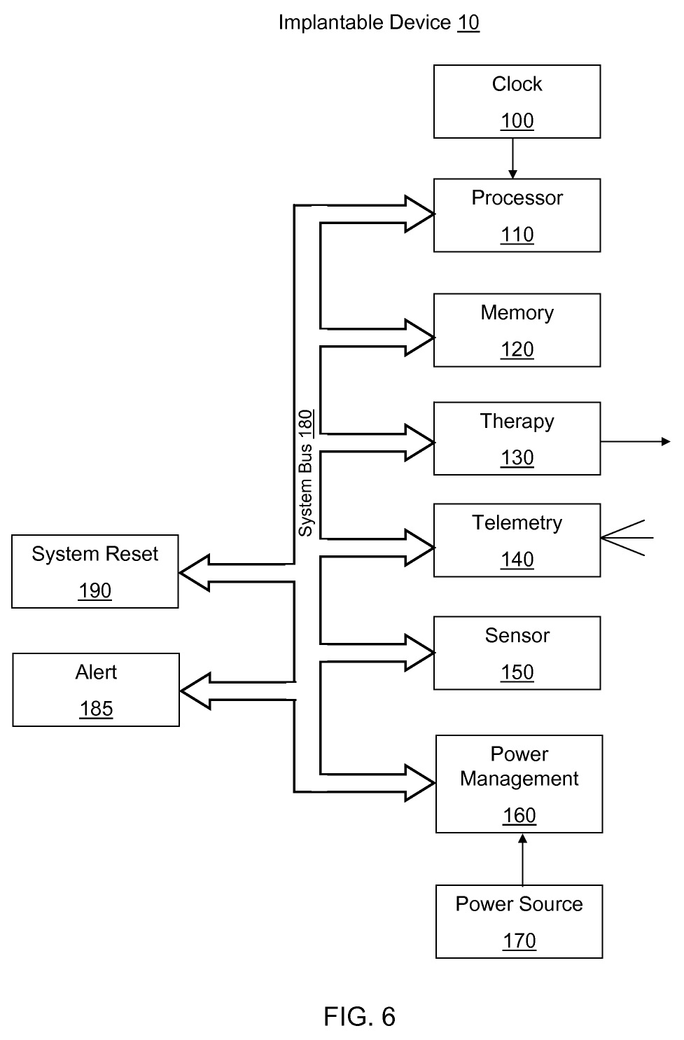

Referring to FIG. 6, some representative electronic components (collectively, “electronics”) of an implantable therapy delivery medical device 10 according to various embodiments are shown in block form. Implantable therapy delivery medical device 10 as depicted in the embodiment shown in FIG. 6 includes a clock 100, a processor 110, a memory 120, a therapy output or delivery component 130, a telemetry component 140, a sensor 150, a power management module 160, a power source 170, an alert module 185, and a system reset module 190. Other components of implantable medical device 10 can include, e.g., a diagnostics module (not shown). All components except the power source 170 can be configured on one or more Application Specific Integrated Circuits (ASICs) or may be one or more discrete components, or a combination of both. Also, all components, except the clock and power source are connected to bi-directional data bus 180 that is non-multiplexed with separate address and data lines.

Processor 110 may be synchronous and typically operates on low power, such as Motorola 68HC11 synthesized core operating with a compatible instruction set. Clock 100 counts the number of seconds since a fixed date for date/time stamping of events and may be used for therapy control. Memory 120 includes memory sufficient for operation of device 10, such as volatile Random Access Memory (RAM) for example static RAM, nonvolatile Read Only Memory (ROM), Electrically Erasable Programmable Read Only Memory (EEPROM) for example Flash EEPROM, and register arrays configured on ASICs. Direct Memory Access (DMA) is available to selected modules such as telemetry module 140 or sensor module 150, so that the selected modules can request control of data bus 180 and write data directly to memory 120 bypassing processor 110. System Reset 190 controls operation of ASICs and modules during power-up of device 10, so ASICs and modules registers can be loaded and brought on-line in a stable condition.

Telemetry 140 module or other wireless module provides for communication between implantable device 10 and external device 40 such as a programmer. Communication may be bi-directional. Telemetry module 140 generally includes a telemetry antenna, a receiver, a transmitter, and a telemetry processor. Telemetry modules are generally known in the art and are further detailed in U.S. Pat. No. 5,752,977, entitled “Efficient High Data Rate Telemetry Format For Implanted Medical Device” issued to Grevious et al. (May 19, 1998), which is incorporate herein by reference in its entirety to the extent that it does not conflict with the disclosure presented herein. While module 140 is referred to herein as “telemetry” module, it will be understood that other forms of wireless communication may readily be substituted where appropriate for telemetry. Examples of forms of wireless communication include Bluetooth®, 802.11, and Medical Implant Communication Service (MICS) frequency band communication.

Therapy module 130 refers to components for carrying out the delivery or generation of therapeutic output to be delivered to a patient from device 10. One of skill in the art will appreciate that the components may vary on a device-by-device basis and a therapy-by-therapy basis. For example, therapy module 130 may contain an oscillator if device 10 is an electrical signal generator and may contain a pumping mechanism and circuitry if device 10 is an infusion device.

Sensor module 150 includes a sensor, e.g. sensors 25, 35 as discussed with regard to FIG. 1, and may include other components for transmitting sensed information from sensor 25, 35 to, e.g., processor 110 or memory 120. Sensor module 150 or other components of device 10 may include one or more analog to digital converters to convert analog signals generated by sensor 25, 35 into digital signals usable by processor 110, as well as suitable filter and amplifier circuitry.

Alert module 185 may issue an alert, e.g. an audible alert or tactile alert, such as a vibration. An alert may be issued if information indicative of an infection is detected. The alert will serve to prompt the patient to seek medical attention.

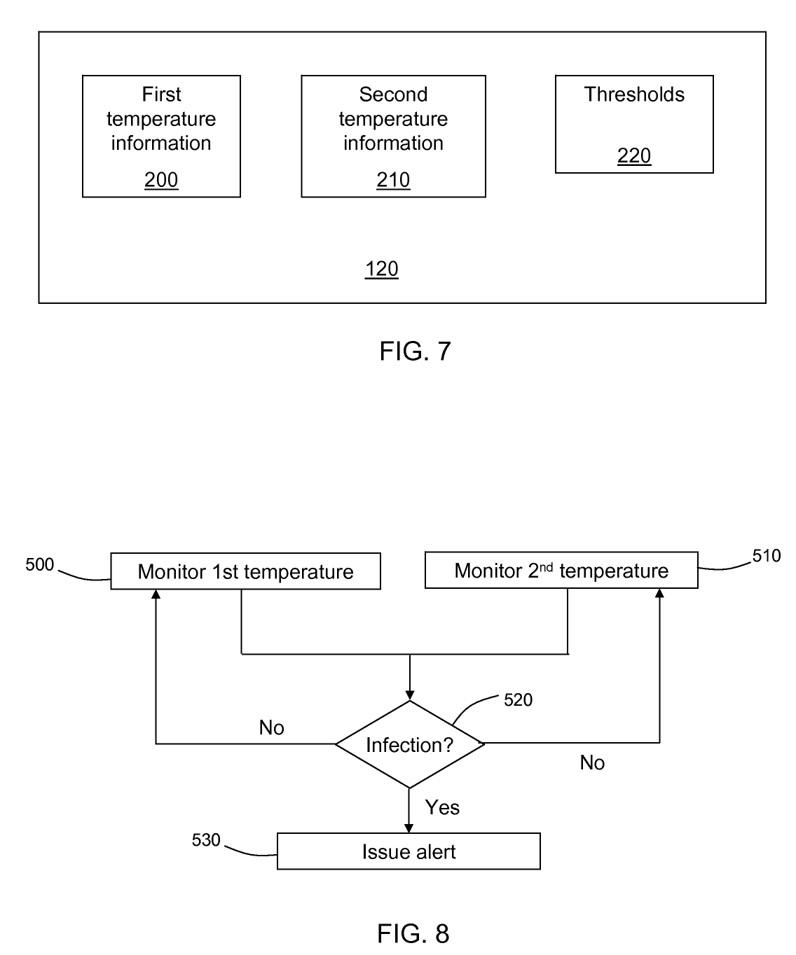

Referring to FIG. 7, memory 120 is shown in more detail. According to various embodiments, memory 120 stores information 200 relating to temperature obtained by first temperature sensor 25 and information 210 relating to temperature obtained by second temperature sensor 35. Memory may also store information regarding threshold values 220. Threshold values may be values specified by an external device 40, such as a physician programmer, and may be specifically tailored to a particular patient. Information stored in memory 120 relating to temperature 200, 210 may be values obtained at a particular point in time, mean or median values, values over time, or the like. Similarly, threshold values 220 may be related to individual absolute values, mean or median values, values over time, or the like. In various embodiments, threshold vales 220 are based on monitored information obtained from first temperature sensor 25 or second temperature sensor 35. In some embodiments, processor 110 compares a value 200 associated with information obtained from first temperature sensor 25 and a value 210 associated with information obtained from second temperature sensor 210 to a look-up table of threshold values 220 stored in memory 120 to determine whether the temperature is indicative of infection or if an action should be taken by device.

In some embodiments, processor 110 may compare information 200, 210 relating to temperature obtained from first and second temperature sensors 25, 35 to calculate threshold values 220 based on the obtained information monitored within the patient. For example, a threshold value 220 may be deviation of 50% or greater, 40% or greater, 30% or greater, 20% or greater, 10% or greater, 5% or greater, etc. from a mean or median value 200, 210 monitored within the patient over a period of time. Processor 110 may compare a temperature metric 200, 210 to a calculated threshold value 220. Of course, in such instances, processor 110 may compare temperature metric 200, 210 to a mean or median temperature metric 200, 210 determined over time to determine whether a threshold has been crossed without first storing such threshold value 220 in memory 120.

It will be understood that the components described in FIGS. 1-7 are but examples of components that an implantable device 10 may have and that many other device or system configurations may be employed to carry out the methods described below. However, for the sake of convenience, the discussion that follows with regard to the methods illustrated in the flow diagrams of FIGS. 8-10 will refer to components as described with regard to FIGS. 1-7.

Referring to FIGS. 8-10, flow diagrams of representative methods of determining whether an infection is in proximity to an implanted therapy delivery device 10 are shown. Generally, the method includes monitoring a first tissue temperature (500) via a first temperature sensor 25 located on a therapy delivery device 10 implanted in a patient 6 and monitoring a second tissue temperature (510) via a second temperature sensor 35 located on an associated therapy delivery element 20 operably coupled to the therapy delivery device 10, the second temperature sensor 35 being at a location removed from the therapy delivery device 10 when in implanted in the patient 6. A determination, based on comparing information relating to the monitored first and second temperatures, is made as to whether a localized infection in proximity to the implantable medical device 10 is likely (520). For example, processor 110 may compare information 200 relating to temperature obtained from first temperature sensor 25 to information 210 relating to temperature obtained from second temperature sensor 35 to threshold values 220 to determine whether a localized infection is likely (520). If an infection is likely, an alert may be issued (530). The alert may include a sensory indication, such as an audible indication or a tactile indication, such as a vibration, or visual indication. A visual indication may include, for example, text or an image. The alert may be issued by implanted device 10 or an external device 40 (see, e.g., FIG. 2), such as a programmer. If the indication is visual, the alert will be presented to the patient or clinician by an external device.

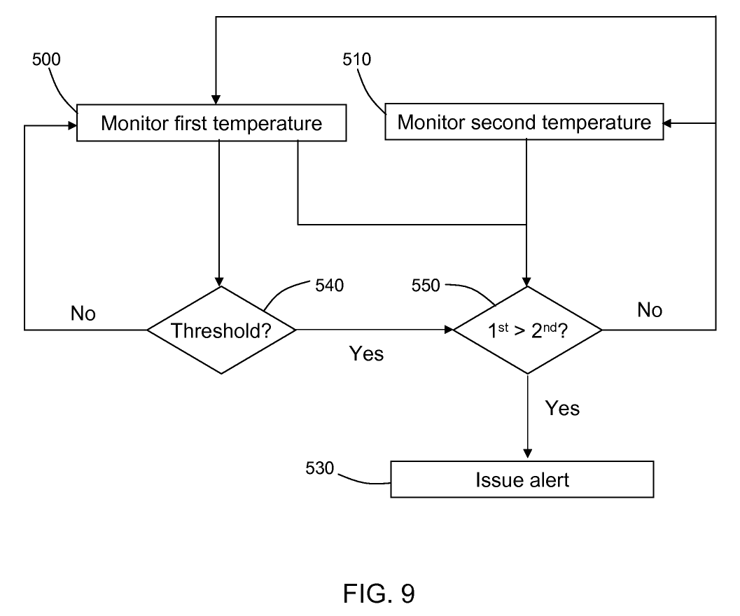

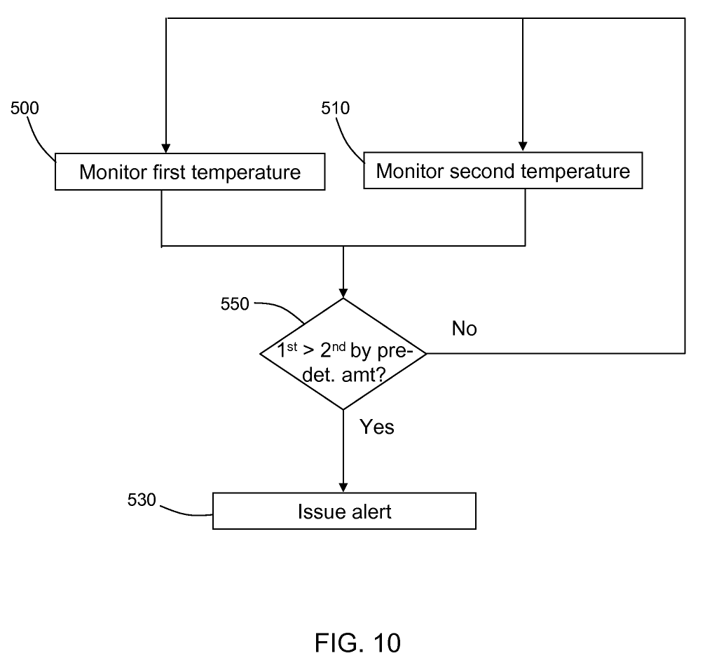

Referring to FIG. 9, a determination as to whether a value associated with the monitored first temperature crosses a threshold (540) is made. If the value does not cross the threshold, then the first temperature may continue to be monitored (500). If the value crosses the threshold, a determination is made as to whether a value associated with the first temperature is greater than a comparable value associated with the second temperature by a predetermined amount (550). If the value associated with the first temperature is greater than the comparable value associated with the second temperature by the predetermined amount, an alert is issued (530). If not, the second temperature may continue to be monitored (510) and the first temperature may continue to be monitored (500). As shown in FIG. 10, determining whether a value associated with the first temperature is greater than a value associated with the second temperature (550) may be sufficient to warrant issuance of an alert (530).

While not shown in FIGS. 8-10, a step of implanting device 1 may be included in the methods described herein.

Thresholds values, against which values associated with monitored first and second temperatures may be compared, will be apparent to skilled practioners or readily obtainable through routine experimentation. For example, a first temperature threshold may be whether the temperature is greater than or equal to 101° F. (38.3 C) at any given point in time, greater than equal to 100.5° F. (38 C) over a 30 minute time period, or the like. By way of further example, a predetermined amount that the first temperature should be greater than the second temperature in order for an alert to be issued may be 2° F. (1.1 C) at a point in time, 1.5° F. (0.8 C) over a 30 minute period of time, or the like.

Additional information regarding use of thresholds determining infection in proximity to an implantable medical device is provided in (i) U.S. patent application Ser. No. ______, entitled “Multi-Parameter Infection Monitoring”, filed on even date herewith, naming Martin Gerber and John Rondoni as inventors, and having P0028531.00 as an attorney docket number; and (ii) U.S. patent application Ser. No. ______, entitled “INDICATOR METRICS FOR INFECTION MONITORING”, filed on even date herewith, naming Martin Gerber and John Rondoni as inventors, and having P0028530.00 as an attorney docket number. The above-referenced patent applications are each hereby incorporated herein by reference in their respective entireties to the extent they do not conflict with the disclosure presented herein.

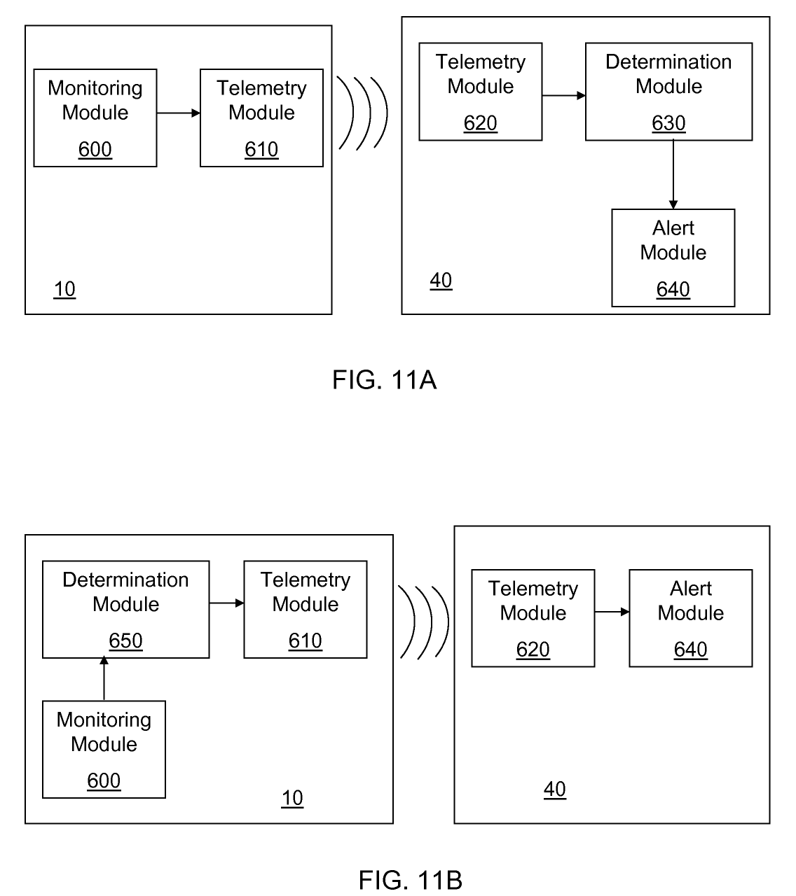

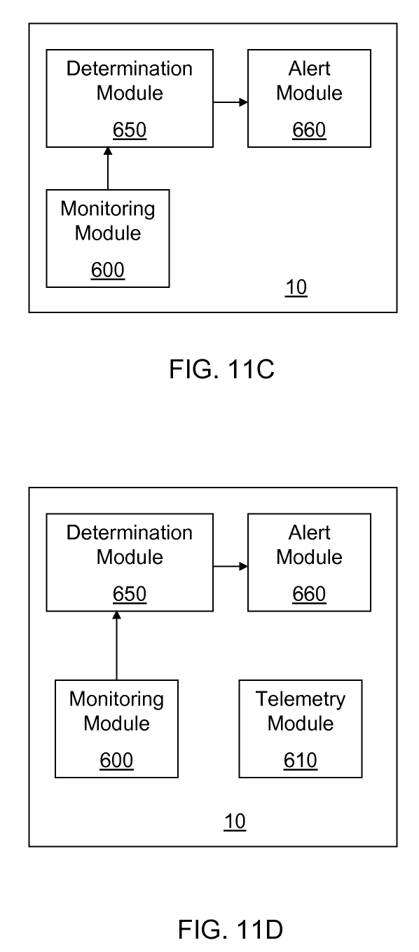

FIGS. 11A-D are block diagrams of representative devices or systems. It will be understood that one or more components described with regard to FIG. 6 may be included or carry out a function of one or more modules described in FIGS. 11A-D. As shown in FIGS. 11A-D, a system or device suitable for carrying out one or more method as discussed with regard to FIGS. 8-10 may include one or more monitoring module 600, telemetry modules 610, 620, a determination module 630, and an alert module 640. Monitoring module 600 allows for sensed information to be provided to electronics of device 10 and may be saved in memory 120. Determination module 630 includes processor 110 that may determine, based on sensed information, whether an infection in proximity to device 10 is likely. If an infection is likely, alert module 640 may be used to issue an alert, e.g. prompting the patient to seek appropriate medical attention. Telemetry modules 610, 620 may be used to communicate information from implanted device 10 to external device 40 (or from external device 40 to internal device 10). As shown in FIGS. 11A-B, certain modules or portions thereof may be in implanted device 10 and certain modules or portions thereof may be in external device 40. As shown in FIGS. 11AC-D, implanted device 10 may include sufficient components to carry out the methods described herein, whether or not device 10 includes a telemetry module 610 for communicating with external device 40.

One of skill in the art will understand that components or steps described herein regarding a given embodiment or set of embodiments may readily be omitted, substituted, or added from, with, or to components or steps of other embodiments or sets of embodiments, as appropriate or desirable.

It will be further understood that a computer readable medium containing instructions that when implemented cause an implantable medical device (or system including an implantable medical device) to perform the methods described herein are contemplated. In an embodiment the electronics comprise a computer readable medium that when implemented cause the device to determine whether a value associated with the first temperature crosses a threshold; determine whether the value associated with the first temperature is greater than a comparable value associated with the second temperature by a predetermined amount; and activate the alert generating module if the value associated with the first temperature crosses the threshold value and is greater than the comparable value associated with the second temperature by the predetermined amount.

In addition, the principles of the methods, systems and devices described herein may be used for detecting various other potential adverse health issues associated with an implantable medical device. For example, temperature may be used to determine whether a hematoma, edema, or seroma is present in proximity to an implanted device. Accordingly, monitoring of such other potential adverse health issues is within the scope of the present disclosure.

Patent applications directed to infection monitoring that may provide additional insight into the teachings provided herein include the following patent applications filed on even date herewith: (i) U.S. patent application Ser. No. ______, entitled “Infection Monitoring”, naming Martin Gerber and John Rondoni as inventors, and having P0028125.00 as an attorney docket number; (ii) U.S. patent application Ser. No. ______, entitled “Controlling Temperature During Recharge for Treatment of Condition”, naming Martin Gerber and John Rondoni as inventors, and having P0028540.00 as an attorney docket number; (iii) U.S. patent application Ser. No. ______, entitled “Event-Triggered Infection Monitoring”, naming Martin Gerber and John Rondoni as inventors, and having P0028528.00 as an attorney docket number; (iv) U.S. patent application Ser. No. ______, entitled “Infection Monitoring”, naming Martin Gerber and John Rondoni as inventors, and having P0028529.00 as an attorney docket number; and (v) U.S. patent application Ser. No. ______, entitled “Refined Infection Monitoring”, naming Martin Gerber and John Rondoni as inventors, and having P0028541.00 as an attorney docket number. The above-referenced patent applications are hereby incorporated herein by reference in their respective entireties to the extent that they do not conflict with the disclosure presented herein.

Thus, embodiments of IMPLANTABLE THERAPY DELIVERY SYSTEM HAVING MULTIPLE TEMPERATURE SENSORS are disclosed. One skilled in the art will appreciate that the present invention can be practiced with embodiments other than those disclosed. The disclosed embodiments are presented for purposes of illustration and not limitation, and the present invention is limited only by the claims that follow.

1. A system comprising:
(i) an implantable medical device comprising:
a housing;
electronics disposed in the housing, the electronics configured to control delivery of therapy; and
a first temperature sensor operably coupled to the electronics and located in proximity to the housing; and
(ii) an implantable therapy delivery element comprising:
a proximal end portion configured to be operably couplable to the implantable medical device, the proximal end portion having a proximal end;
a distal end portion configured to be placed in a patient at a location removed from the implantable medical device, the distal end portion having a distal end; and
a second temperature sensor located on the element between the proximal end and the distal end, the second temperature sensor being operably couplable to the electronics,
wherein the electronics are further configured to compare a value associated with the first temperature sensed by the first temperature sensor to a value associated with the second temperature sensed by the second temperature sensor.

2. The system of claim 1, wherein the implantable medical device further comprises a first electrical contact operably coupled to the electronics, and wherein the implantable therapy delivery element comprises a second electrical contact configured to contact the first electrical contact when the implantable therapy element is coupled to the implantable medical device, the second electrical contact being electrically coupled to the second temperature sensor.

3. The system of claim 1, wherein the electronics comprise an alert generating module.

4. The system of claim 3, wherein the electronics comprise a computer readable medium that when implemented cause the device to determine whether a value associated with the first temperature crosses a threshold; determine whether the value associated with the first temperature is greater than a comparable value associated with the second temperature by a predetermined amount; and activate the alert generating module if the value associated with the first temperature crosses the threshold value and is greater than the comparable value associated with the second temperature by the predetermined amount.

5. The system of claim 1, wherein the implantable medical device is an implantable electrical signal generator and wherein the therapy delivery element is an implantable medical lead.

6. The system of claim 1, wherein the implantable medical device is an implantable electrical signal generator and wherein the therapy delivery element is a lead extension.

7. The system of claim 1, wherein the implantable medical device is an implantable infusion device and wherein the therapy delivery element is an implantable catheter.

8. A method for monitoring an infection in proximity to a therapy delivering medical device implanted in a patient, the therapy delivering medical device being part of a system comprising an associated therapy element for delivering the therapy from the therapy delivering device to the patient, the therapy delivery element being operably couplable to the therapy delivering medical device, the method comprising:
monitoring a first patient tissue temperature via a first temperature sensor that is a part of the therapy delivering implanted medical device;
monitoring a second patient tissue temperature via a second temperature sensor that is a part of the associated therapy element at a location removed from the therapy delivering implanted medical device;
determining whether an infection is present in proximity to the therapy delivering medical device by comparing information relating to the first monitored temperature to information relating to the second monitored temperature; and
issuing an alert if a determination is made that an infection is present in proximity to the therapy delivering medical device.

9. The method of claim 8, wherein determining whether an infection is present in proximity to the therapy delivering medical device comprises determining whether a value associated with the first temperature is greater than a comparable value associated with the second temperature by a predetermined amount.

10. The method of claim 9, wherein determining whether an infection is present in proximity to the therapy delivering medical device further comprises:
determining whether a value associated with the first temperature crosses a threshold.

11. The method of claim 8, wherein the therapy delivering medical device is an implantable electrical signal generator and the associated therapy element is an implantable medical lead.

12. The method of claim 8, wherein the therapy delivering medical device is an implantable electrical signal generator and the associated therapy element is an implantable medical lead extension.

13. The method of claim 8, wherein the therapy delivering medical device is an implantable infusion device and the associated therapy element is an implantable catheter.

14. The method of claim 8, wherein the determination of whether an infection is present in proximity to the therapy delivering medical device is performed by the therapy delivering medical device.

15. The method of claim 8, wherein issuing the alert comprises activating an alert module of the therapy delivering medical device.

16. The method of claim 8, wherein the determination of whether an infection is present in proximity to the therapy delivering medical device is performed by an external device in wireless communication with the therapy delivering medical device.

17. The method of claim 8, wherein issuing the alert comprises activating an alert module of an external device in wireless communication with the therapy delivering medical device.

18. An implantable medical device comprising:
a housing;
electronics disposed in the housing, the electronics configured to control delivery of therapy; and
a first temperature sensor operably coupled to the electronics and located in proximity to the housing.
wherein the electronics are further configured to compare a value associated with the first temperature sensed by the first temperature sensor to a value associated with a second temperature sensor located on an implantable therapy delivery element operably couplable to the electronics.

19. The device of claim 18, wherein the first temperature sensor is disposed on the housing of the implantable medical device.

20. The system of claim 1, wherein the first temperature sensor is disposed on the housing of the implantable medical device.
